(19) United States
(12) Patent Application Publication
(10) Pub. No.: US 2025/0177720 A1
(43) Pub. Date: Jun. 5, 2025

(54) MEDICAL TUBING BREAKAWAY DEVICE AND METHODS OF USE

(71) Applicant: University of Washington, Seattle, WA (US)

(72) Inventors: Liam Sullivan, Seattle, WA (US); Michael Malone, Seattle, WA (US); Joelle Tudor, Seattle, WA (US); Emmanuel Pacheco, Seattle, WA (US); Austin Higgs, Seattle, WA (US); Tova Weiss, Seattle, WA (US)

(21) Appl. No.: 18/842,977

(22) PCT Filed: Mar. 2, 2023
(86) PCT No.: PCT/US2023/063545

§ 371 Date: Aug. 30, 2024

Related U.S. Application Data
(60) Provisional application No. 63/315,822, filed on Mar. 2, 2022.

(51) Int. Cl. A61M 39/20

(52) U.S. Cl. CPC A61M 39/20

(57) ABSTRACT

The present disclosure provides a device comprising a first connector including a first elongated hollow member configured to be positioned at least partially within a lumen of a proximal portion of a tube. The first connector provides a clamping force to the proximal portion of the tube. The device also includes a second connector configured to be removably coupled to the first connector. The second connector including a second elongated hollow member configured to be positioned at least partially within a lumen of a distal portion of the tube. The proximal portion of the tube is separated from the distal portion of the tube when the first connector is disconnected from the second connector, and the lumen of the proximal portion of the tube is fluidically connected with the lumen of the distal portion of the tube when the first connector is removably coupled to the second connector.

### CROSS-REFERENCE TO RELATED APPLICATIONS

This application claims priority to U.S. Provisional Patent Application No. 63/315,822, filed Mar. 2, 2022, the contents of which are hereby incorporated by reference in their entirety.

### BACKGROUND

Unless otherwise indicated herein, the materials described in this section are not prior art to the claims in this application and are not admitted to be prior art by inclusion in this section.

Various medical tubing are commonly used in a variety of medical procedures. Such tubing includes, as non-limiting examples, urinary catheters, intravenous (IV) lines, spinal tap tubing, nasogastric (NG) tube, suprapubic catheters, and gastrostomy tubes. Common concerns about such medical tubing is the pain and trauma to the patient when the tubing is pulled (either accidentally or intentionally by the patient) prior to its desired removal.

### SUMMARY

The present disclosure provides a medical tubing breakaway device that reduces the risk of trauma to the patient when the medical tubing is removed prematurely. In one non-limiting example, the medical tubing comprises a urinary catheter. Urinary catheters are commonly used as indwelling catheters that have soft, plastic or rubber tube inserted into the bladder to drain urine from the patient. A urinary catheter forms continuous urine flow from a patient's bladder to a urine collection bag through a urine channel. The device of the present disclosure is inserted into the urine lumen of a cut urinary catheter just external to the body after the urinary catheter has been placed. The device keeps the urinary balloon inflated and allows urine to flow as normal. When the external tubing is pulled, the external tube disconnects at the device interface, leaving the balloon safely in place and preventing trauma to the patient. The two parts can then be easily reconnected to resume regular catheter function. After the intact urinary catheter tube is sliced and the device is completely installed into either end, urine will flow continuously across the disconnect, the inflation lumen will be shut to prevent the balloon from deflating, and the device will disconnect when a certain pull-force is reached.

In particular, in a first aspect, the present disclosure provides a device comprising a first connector including a first elongated hollow member configured to be positioned at least partially within a lumen of a proximal portion of a tube. The first connector provides a clamping force to the proximal portion of the tube. The device also includes a second connector configured to be removably coupled to the first connector. The second connector includes a second elongated hollow member configured to be positioned at least partially within a lumen of a distal portion of the tube. The second connector provides a clamping force to the distal portion of the tube. The proximal portion of the tube is separated from the distal portion of the tube when the first connector is disconnected from the second connector, and the lumen of the proximal portion of the tube is fluidically connected with the lumen of the distal portion of the tube when the first connector is removably coupled to the second connector.

In a second aspect, the present disclosure provides a kit comprising (a) the device of the first aspect, and (b) a urinary catheter comprising: (i) a tube having a distal end and a proximal end opposite the distal end, wherein the tube includes an inflation lumen and a drainage lumen, and (ii) a balloon positioned near the proximal end of the tube, wherein the inflation lumen is in communication with an interior of the balloon.

In a third aspect, the present disclosure provides a kit comprising (a) the device of the first aspect, and a drainage cap configured to be removably coupled to the first connector, wherein the drainage cap prevents fluid from exiting the lumen of the proximal portion of the tube through the first elongated hollow member.

In a fourth aspect, the present disclosure provides a device including a first elongated hollow member configured to be positioned at least partially within a lumen of a proximal portion of a tube. The device also includes a clamping mechanism configured to provide a clamping force to the proximal portion of the tube. The device also includes a second elongated hollow member configured to be positioned at least partially within a lumen of a distal portion of the tube. The second elongated hollow member and the first elongated hollow member are fluidically connected such that the lumen of the proximal portion of the tube is fluidically connected with the lumen of the distal portion of the tube. A force required to remove the first elongated hollow member from the lumen of the proximal portion of the tube is greater than a force required to remove the second elongated hollow member from the lumen of the distal portion of the tube.

In a fifth aspect, the present disclosure provides a kit comprising (a) the device of the fourth aspect, and (b) a urinary catheter comprising: (i) a tube having a distal end and a proximal end opposite the distal end, wherein the tube includes an inflation lumen and a drainage lumen, and (ii) a balloon positioned near the proximal end of the tube, wherein the inflation lumen is in communication with an interior of the balloon.

In a sixth aspect, the present disclosure provides a method for installing a urinary catheter breakaway device, the method comprising: (a) cutting a urinary catheter between a proximal end and a distal end of the urinary catheter to thereby define a proximal portion and a distal portion of the urinary catheter, (b) positioning a first elongated hollow member of a first connector at least partially within a lumen of the proximal portion of the urinary catheter, (c) positioning a second elongated hollow member of a second connector at least partially within a lumen of the distal portion of the urinary catheter, (d) providing a clamping force to the proximal portion of the urinary catheter, (e) providing a clamping force to the distal portion of the urinary catheter, and (f) removably coupling the first connector to the second connector, wherein the lumen of the proximal portion of the urinary catheter is fluidically connected with the lumen of the distal portion of the urinary catheter when the first connector is removably coupled to the second connector.

In a seventh aspect, the present disclosure provides a method for installing a urinary catheter breakaway device, the method comprising: (a) cutting a urinary catheter between a proximal end and a distal end of the urinary catheter to thereby define a proximal portion and a distal portion of the urinary catheter, (b) positioning a first elongated hollow member of the urinary catheter breakaway device at least partially within a lumen of the proximal portion of the urinary catheter, (c) positioning a second elongated hollow member of the urinary catheter breakaway device at least partially within a lumen of the distal portion of the urinary catheter, (d) providing a clamping force to the proximal portion of the urinary catheter such that a force required to remove the first elongated hollow member from the lumen of the proximal portion of the urinary catheter is greater than a force required to remove the second elongated hollow member from the lumen of the distal portion of the urinary catheter.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates an example device with a first connector removably coupled to a second connector, according to an example embodiment.

FIG. 2 illustrates a close up view of the device of FIG. 1, according to an example embodiment.

FIG. 3 illustrates the device of FIG. 1 with the first connector separated from the second connector, according to an example embodiment.

FIG. 4 illustrates a close up view of the device of FIG. 3, according to an example embodiment.

FIG. 5 illustrates a perspective cross-sectional view of an example device, according to an example embodiment.

FIG. 6 illustrates a perspective view of the device of FIG. 5, according to an example embodiment.

FIG. 7 illustrates a perspective view of an example first connector, according to an example embodiment.

FIG. 8 illustrates a perspective view of an example second connector, according to an example embodiment.

FIG. 9 illustrates a side cross-sectional view of an example device, according to an example embodiment.

FIG. 10 illustrates a side cross-sectional view of the device of FIG. 9 including a first cap and a second cap, according to an example embodiment.

FIG. 11 illustrates a side view of the device of FIG. 10, according to an example embodiment.

FIG. 12 illustrates a perspective view of an example cap, according to an example embodiment.

FIG. 13 illustrates a side cross-sectional view of the cap of FIG. 12, according to an example embodiment.

FIG. 14 illustrates a perspective view of an example connector, according to an example embodiment.

FIG. 15 illustrates a perspective view of an example cap, according to an example embodiment.

FIG. 16 illustrates a side cross-sectional view of the cap of FIG. 15, according to an example embodiment.

FIG. 17 illustrates a side cross-sectional view of a drainage cap removably coupled to the first connector, according to an example embodiment.

FIG. 18 illustrates a perspective view of the drainage cap of FIG. 17, according to an example embodiment.

FIG. 19 is a side cross-sectional view of another example device, according to an example embodiment.

FIG. 20 is a flowchart illustrating an example method for installing a urinary catheter breakaway device, according to an example embodiment.

FIG. 21 is a flowchart illustrating another example method for installing a urinary catheter breakaway device, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the Figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

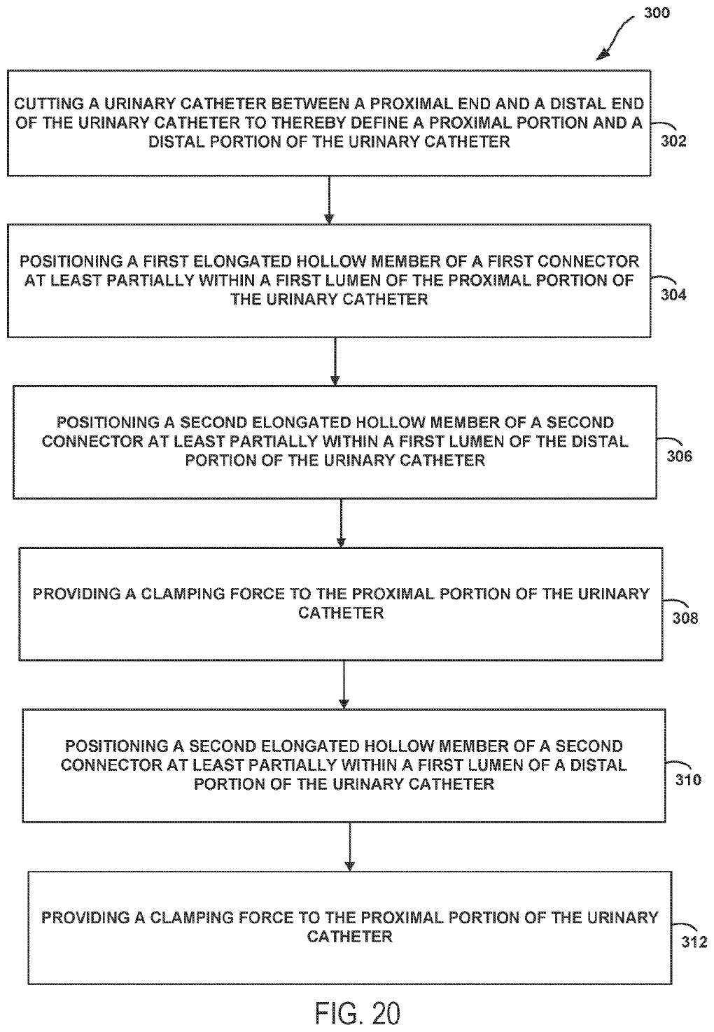

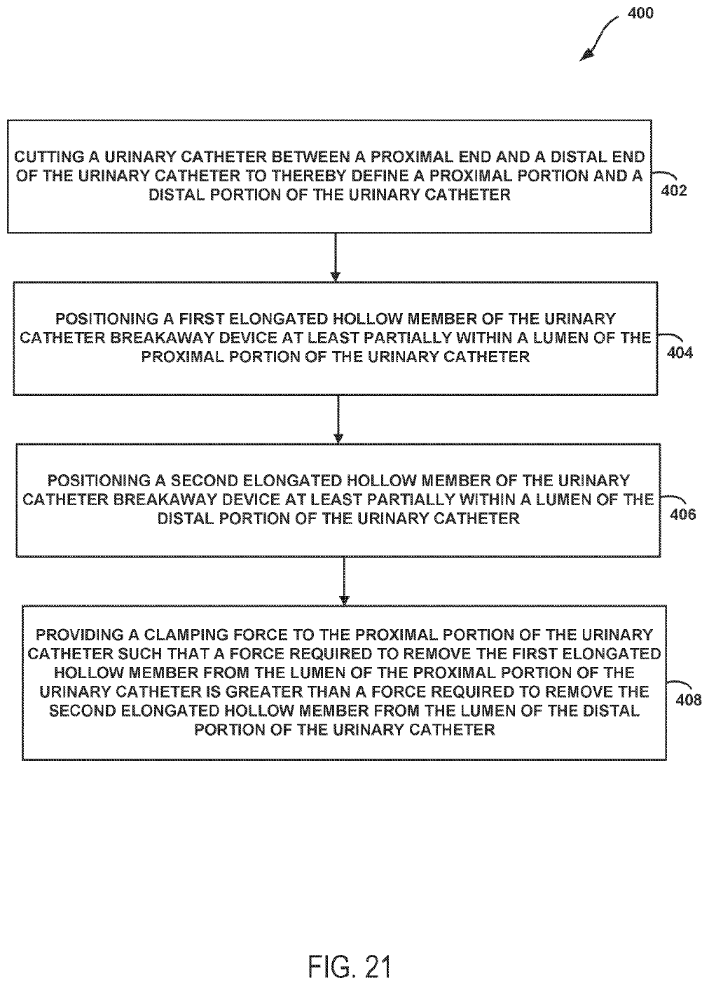

In FIGS. 20-21, referred to above, the blocks may represent operations and/or portions thereof and lines connecting the various blocks do not imply any particular order or dependency of the operations or portions thereof. It will be understood that not all dependencies among the various disclosed operations are necessarily represented. FIGS. 20-21 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Reference to, e.g., a “first” item does not require or preclude the existence of, e.g., a “second” or higher-numbered item. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements, “about” means+/−5%.

As used herein, with respect to measurements, “substantially” means+/−5%.

Generally, the present disclosure provides a supplement to a medical tubing to minimize, limit, prevent or eliminate the risk of trauma to the patient when the medical tubing is pulled prior to desired removal. The device is inserted into a lumen of a cut medical tubing external to the body after the medical tubing has been placed. The device keeps the lumen open to thereby allow liquid to flow through the medical tubing as normal. When the tubing is pulled, the tube disconnects at the device interface, thereby preventing trauma to the patient. The two parts of the device can then be easily reconnected to resume regular function of the medical tubing.

Thus, according to an embodiment, the present disclosure provides a device comprising a first connector including a first elongated hollow member configured to be positioned at least partially within a lumen of a proximal portion of a tube. The first connector provides a clamping force to the proximal portion of the tube. The device also includes a second connector configured to be removably coupled to the first connector. The second connector includes a second elongated hollow member configured to be positioned at least partially within a lumen of a distal portion of the tube. The second connector provides a clamping force to the distal portion of the tube. The proximal portion of the tube is separated from the distal portion of the tube when the first connector is disconnected from the second connector, and the lumen of the proximal portion of the tube is fluidically connected with the lumen of the distal portion of the tube when the first connector is removably coupled to the second connector.

In an embodiment, the second elongated hollow member comprises a first portion configured to be positioned at least partially within the lumen of the distal portion of the tube, and the second elongated hollow member comprises a second portion configured to be positioned at least partially within the first elongated hollow member when the first connector is removably coupled to the second connector.

In an embodiment, the first elongated hollow member comprises a first portion configured to be positioned at least partially within the lumen of the proximal portion of the tube, and the first elongated hollow member comprises a second portion configured to be positioned at least partially within the second elongated hollow member when the first connector is removably coupled to the second connector.

In an embodiment, the first connector and the second connector interact to provide a watertight seal between the first connector and the second connector when the first connector is removably coupled to the second connector.

In an embodiment, the first elongated hollow member includes one or more ridges extending away from an exterior surface of the first elongated hollow member, and the second elongated hollow member includes one or more ridges extending away from an exterior surface of the second elongated hollow member.

In an embodiment, the first connector is removably coupled to the second connector via a magnetic connection.

In an embodiment, the first connector includes one or more first magnets, and wherein the second connector includes one or more second magnets configured to interact with the one or more first magnets to thereby removably couple the first connector to the second connector.

In an embodiment, the first connector is removably coupled to the second connector via a mechanical connection.

In an embodiment, the first connector includes a plurality of arms, and wherein the second connector includes a coupling mechanism with which the plurality of arms are configured to interact to thereby removably couple the first connector to the second connector.

In an embodiment, the clamping force to the proximal portion of the tube occludes a second lumen of the proximal portion of the tube.

In an embodiment, the clamping force to the distal portion of the tube occludes a second lumen of the distal portion of the tube.

In an embodiment, the first connector includes: (a) a first plurality of arms positioned circumferentially around the first elongated hollow member, and (b) a first cap positioned over the first plurality of arms, and the second connector includes: (a) a second plurality of arms positioned circumferentially around the second elongated hollow member, and (b) a second cap positioned over the second plurality of arms.

In an embodiment, the first plurality of arms are configured to transition from a first position to a second position when the first cap moves with respect to the first plurality of arms, the first plurality of arms are configured to provide the clamping force to the proximal portion of the tube when the first plurality of arms are in the second position, the second plurality of arms are configured to transition from a first position to a second position when the second cap moves with respect to the second plurality of arms, and the second plurality of arms are configured to provide the clamping force to the distal portion of the tube when the second plurality of arms are in the second position.

In an embodiment, each of the first plurality of arms includes a first indent and a second indent, the first cap includes a protrusion configured to mate with the first indent when the first plurality of arms are in the first position, the protrusion is configured to mate with the second indent when the first plurality of arms are in the second position, each of the second plurality of arms includes a first indent and a second indent, the second cap includes a protrusion configured to mate with the first indent when the second plurality of arms are in the first position, and the protrusion is configured to mate with the second indent when the second plurality of arms are in the second position.

In an embodiment, the first plurality of arms are locked in the second position once the first plurality of arms transition from the first position to the second position, and the second plurality of arms are locked in the second position once the second plurality of arms transition from the first position to the second position.

In an embodiment, the first plurality of arms can transition from the second position to the first position to thereby release the clamping force to the proximal portion of the tube, and the second plurality of arms can transition from the second position to the first position to thereby release the clamping force to the distal portion of the tube.

In an embodiment, each of the first plurality of arms includes a first ramp, wherein the first cap includes a second ramp, a rotation of the first cap with respect to the first plurality of arms causes the first ramp to interact with the second ramp to thereby transition the first plurality of arms from the second position to the first position, each of the second plurality of arms includes a first ramp, the second cap includes a second ramp, and a rotation of the second cap with respect to the second plurality of arms causes the first ramp to interact with the second ramp to thereby transition the second plurality of arms from the second position to the first position.

In an embodiment, an inner diameter of the first elongated hollow member is constant and is within about 30% of an inner diameter of the lumen of the proximal portion of the tube, and an inner diameter of the second elongated hollow member is constant and is within about 30% of an inner diameter of the lumen of the distal portion of the tube.

In an embodiment, the tube comprises one of a urinary catheter, an intravenous (IV) line, a spinal tap tubing, a nasogastric (NG) tube, a suprapubic catheter, or a gastrostomy tube.

In an embodiment, the clamping force to the proximal portion of the tube is symmetrical with respect to a longitudinal axis of the tube.

In an embodiment, the clamping force to the distal portion of the tube is symmetrical with respect to a longitudinal axis of the tube.

In an embodiment, the device includes a flow prevention mechanism positioned in the first connector, and the flow prevention mechanism is configured to prevent a fluid from exiting the first hollow elongated member when the first connector is disconnected from the second connector.

According to another embodiment, the present disclosure provides a kit comprising: (a) the device of any one of the embodiments described above, and (b) a urinary catheter, comprising: (i) a tube having a distal end and a proximal end opposite the distal end, wherein the tube includes an inflation lumen and a drainage lumen, and (ii) a balloon positioned near the proximal end of the tube, wherein the inflation lumen is in communication with an interior of the balloon.

According to another embodiment, the present disclosure provides a kit comprising: (a) the device of any one of the embodiments described above, and (b) a drainage cap configured to be removably coupled to the first connector, wherein the drainage cap prevents fluid from exiting the lumen of the proximal portion of the tube through the first elongated hollow member.

In an embodiment, the drainage cap is removably coupled to the first connector via a magnetic connection.

In an embodiment, the first connector includes one or more first magnets, and wherein the drainage cap includes one or more second magnets configured to interact with the one or more first magnets to thereby removably couple the first connector to the drainage cap.

In an embodiment, the drainage cap is removably coupled to the first connector via a mechanical connection.

In an embodiment, the first connector includes a plurality of arms, and wherein the drainage cap includes a coupling mechanism with which the plurality of arms are configured to interact to thereby removably couple the first connector to the drainage cap.

According to another embodiment, the present disclosure provides a device including a first elongated hollow member configured to be positioned at least partially within a lumen of a proximal portion of a tube. The device also includes a clamping mechanism configured to provide a clamping force to the proximal portion of the tube. The device also includes a second elongated hollow member configured to be positioned at least partially within a lumen of a distal portion of the tube. The second elongated hollow member and the first elongated hollow member are fluidically connected such that the lumen of the proximal portion of the tube is fluidically connected with the lumen of the distal portion of the tube. A force required to remove the first elongated hollow member from the lumen of the proximal portion of the tube is greater than a force required to remove the second elongated hollow member from the lumen of the distal portion of the tube.

In an embodiment, the first elongated hollow member includes one or more ridges extending away from an exterior surface of the first elongated hollow member, and the second elongated hollow member includes one or more ridges extending away from an exterior surface of the second elongated hollow member.

In an embodiment, the clamping force to the proximal portion of the tube occludes a second lumen of the proximal portion of the tube.

In an embodiment, the clamping force to the proximal portion of the tube is symmetrical with respect to a longitudinal axis of the tube.

In an embodiment, a diameter of the first elongated hollow member is constant and is within about 30% of a diameter of the lumen of the proximal portion of the tube, and wherein a diameter of the second elongated hollow member is constant and is within about 30% of a diameter of the lumen of the distal portion of the tube.

In an embodiment, the clamping mechanism comprises: a plurality of arms positioned circumferentially around the first elongated hollow member, and a cap positioned over the plurality of arms.

In an embodiment, the plurality of arms are configured to transition from a first position to a second position when the cap moves with respect to the plurality of arms, and plurality of arms are configured to provide the clamping force to the proximal portion of the tube when the plurality of arms are in the second position.

In an embodiment, each of the plurality of arms includes a first indent and a second indent, the cap includes a protrusion configured to mate with the first indent when the plurality of arms are in the first position, and the protrusion is configured to mate with the second indent when the plurality of arms are in the second position.

In an embodiment, the plurality of arms are locked in the second position once the plurality of arms transition from the first position to the second position.

In an embodiment, the plurality of arms can transition from the second position to the first position to thereby release the clamping force to the proximal portion of the tube.

According to another embodiment, the present disclosure provides a kit comprising: (a) the device of any one of the embodiments described above, and (b) a urinary catheter, comprising: (i) a tube having a distal end and a proximal end opposite the distal end, wherein the tube includes an inflation lumen and a drainage lumen, and (ii) a balloon positioned near the proximal end of the tube, wherein the inflation lumen is in communication with an interior of the balloon.

According to another embodiment, the present disclosure provides a method for installing a urinary catheter breakaway device, the method comprising: (a) cutting a urinary catheter between a proximal end and a distal end of the urinary catheter to thereby define a proximal portion and a distal portion of the urinary catheter, (b) positioning a first elongated hollow member of a first connector at least partially within a lumen of the proximal portion of the urinary catheter, (c) positioning a second elongated hollow member of a second connector at least partially within a lumen of the distal portion of the urinary catheter, (d) providing a clamping force to the proximal portion of the urinary catheter, (e) providing a clamping force to the distal portion of the urinary catheter, and (f) removably coupling the first connector to the second connector, wherein the lumen of the proximal portion of the urinary catheter is fluidically connected with the lumen of the distal portion of the urinary catheter when the first connector is removably coupled to the second connector.

In an embodiment, the method further includes applying a pinching force to the proximal portion of the urinary catheter and the distal portion of the urinary catheter prior to cutting the urinary catheter, and removing the pinching force after removably coupling the first connector to the second connector.

In an embodiment, the clamping force to the proximal portion of the urinary catheter occludes a second lumen of the proximal portion of the urinary catheter, and the clamping force to the distal portion of the urinary catheter occludes a second lumen of the distal portion of the urinary catheter.

According to another embodiment, the present disclosure provides a method for installing a urinary catheter breakaway device, the method comprising: (a) cutting a urinary catheter between a proximal end and a distal end of the urinary catheter to thereby define a proximal portion and a distal portion of the urinary catheter, (b) positioning a first elongated hollow member of the urinary catheter breakaway device at least partially within a lumen of the proximal portion of the urinary catheter, (c) positioning a second elongated hollow member of the urinary catheter breakaway device at least partially within a lumen of the distal portion of the urinary catheter, (d) providing a clamping force to the proximal portion of the urinary catheter such that a force required to remove the first elongated hollow member from the lumen of the proximal portion of the urinary catheter is greater than a force required to remove the second elongated hollow member from the lumen of the distal portion of the urinary catheter.

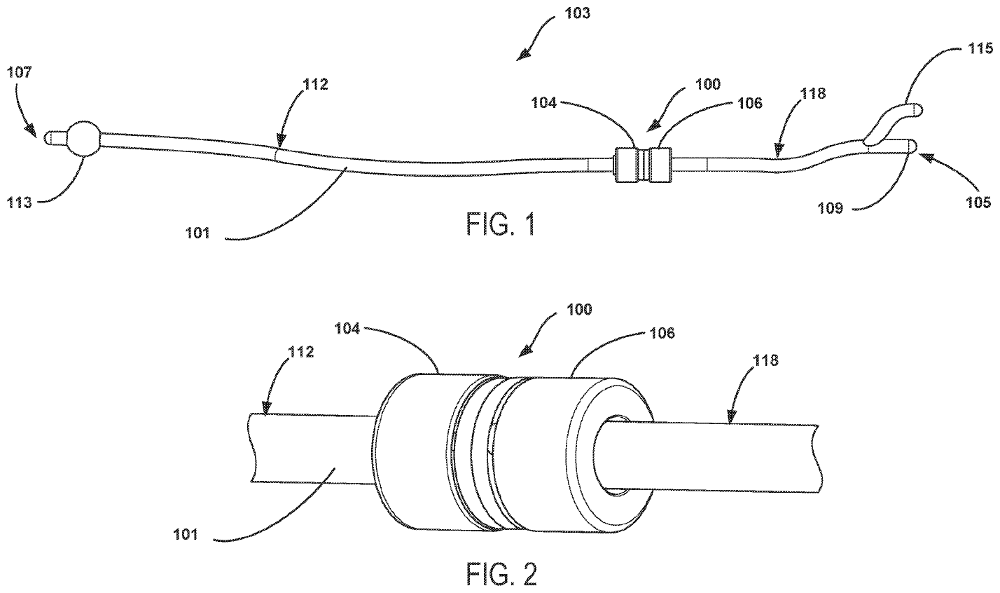

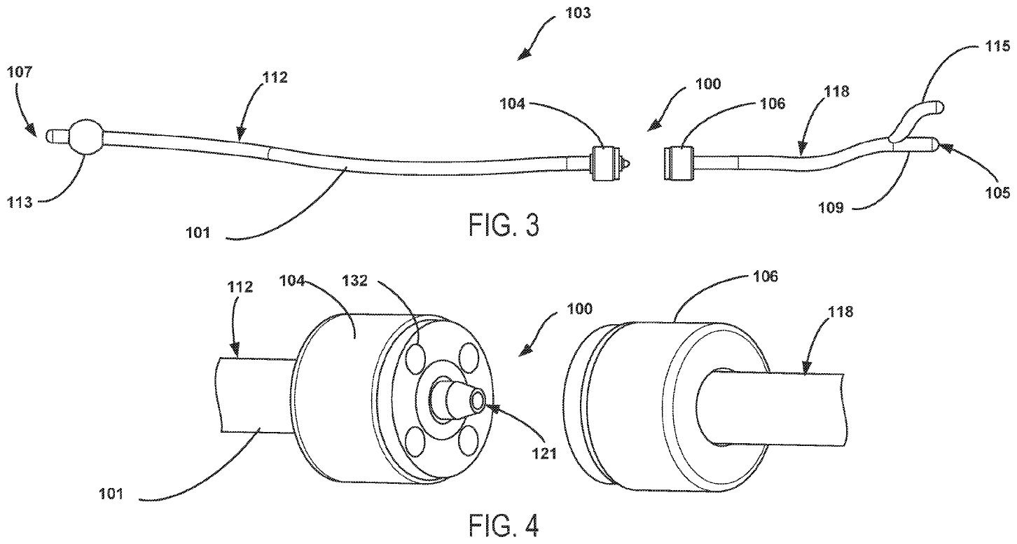

With reference to the Figures, the present disclosure provides a device 100 designed to minimize, limit, prevent or eliminate the risk of trauma to the patient when a tube 101 is pulled prior to desired removal. The device 100 includes a first connector 104 and a second connector 106 each inserted into the tube 101 external to the body after the tube 101 has been placed. FIGS. 1-4 illustrate an example tube 101 comprising a urinary catheter 103. Such a urinary catheter 103 includes a lumen 110 (e.g., a drainage lumen), a second lumen 111 (e.g., an inflation lumen), and a balloon 113 positioned near the proximal end 107 of the tube 101, and the second lumen 111 is in communication with an interior of the balloon 113. Although a urinary catheter 103 is shown in FIGS. 1-4, other medical tubing would benefit from the design of the device 100 described herein, such as an intravenous (IV) line, a spinal tap tubing, a nasogastric (NG) tube, a suprapubic catheter, or a gastrostomy tube, as non-limiting examples. The device 100 is designed to keep a lumen of the tube 101 open when the first connector 104 is coupled to the second connector 106 to thereby allow liquid to flow through the tube 101 as normal when the first connector 104 is coupled to the second connector 106. When the tube 101 is pulled, the tube 101 disconnects at the interface between the first connector 104 and the second connector 106 as shown in FIGS. 3 and 4, thereby preventing trauma to the patient. The first connector 104 and the second connector 106 of the device 100 can then be easily reconnected to resume regular function of the tube 101, as shown in FIGS. 1 and 2.

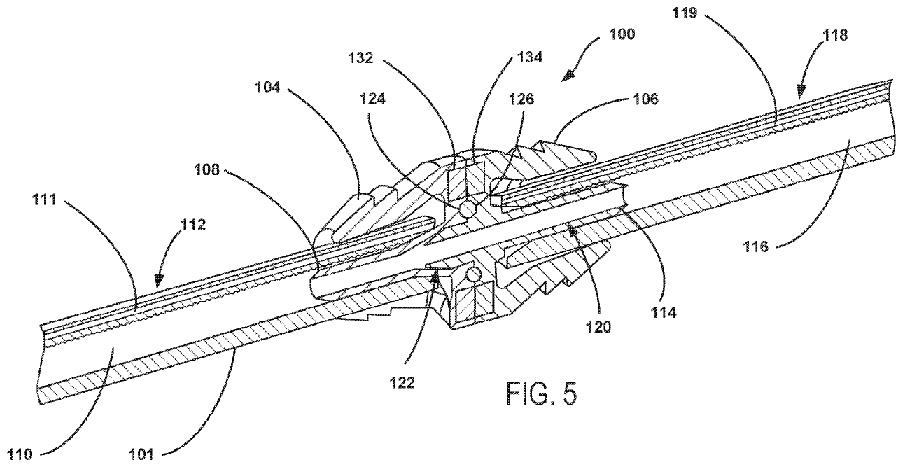

In particular, with reference to FIG. 5, the present disclosure provides a device 100 comprising a first connector 104 including a first elongated hollow member 108 configured to be positioned at least partially within a lumen 110 of a proximal portion 112 of a tube 101. The first connector 104 provides a clamping force to the proximal portion 112 of the tube 101. In one particular example, the first connector 104 provides the clamping force to an outer surface of the tube 101. In such an example, the first elongated hollow member 108 may be rigid such that the clamping force is applied circumferentially around the first elongated hollow member 108. Even when the clamping force is applied, the first elongated hollow member 108 maintains fluid connectivity with the lumen 110 of the proximal portion 112 of the tube 101. The device 100 also includes a second connector 106 configured to be removably coupled to the first connector 104. The second connector 106 includes a second elongated hollow member 114 configured to be positioned at least partially within a lumen 116 of a distal portion 118 of the tube 101. The second connector 106 provides a clamping force to the distal portion 118 of the tube 101. In one particular example, the second connector 106 provides the clamping force to an outer surface of the tube 101. Similar to the first elongated hollow member 108, in one example the second elongated hollow member 114 may be rigid such that the clamping force is applied circumferentially around the second elongated hollow member 114. Even when the clamping force is applied, the second elongated hollow member 114 maintains fluid connectivity with the lumen 116 of the distal portion 118 of the tube 101. The proximal portion 112 of the tube 101 is separated from the distal portion 118 of the tube 101 when the first connector 104 is disconnected from the second connector 106, and the lumen 110 of the proximal portion 112 of the tube 101 is fluidically connected with the lumen 116 of the distal portion 118 of the tube 101 when the first connector 104 is removably coupled to the second connector 106.

In one example, as shown in FIG. 5, the second elongated hollow member 114 comprises a first portion 120 configured to be positioned at least partially within the lumen 110 of the distal portion 118 of the tube 101, and the second elongated hollow member 114 comprises a second portion 122 configured to be positioned at least partially within the first elongated hollow member 108 when the first connector 104 is removably coupled to the second connector 106. In another example, as shown in FIG. 4, an inverse to the configuration described above in relation to FIG. 5 is also possible. In particular, the first elongated hollow member 108 comprises a first portion configured to be positioned at least partially within the lumen 110 of the proximal portion 112 of the tube 101, and the first elongated hollow member 108 comprises a second portion 121 configured to be positioned at least partially within the second elongated hollow member 114 when the first connector 104 is removably coupled to the second connector 106. In yet another example, the first elongated hollow member 108 and the second elongated hollow member 114 abut one another and do not overlap with each other at all.

In one example, an inner diameter of the first elongated hollow member 108 is constant and is within about 30% of an inner diameter of the lumen 110 of the proximal portion 112 of the tube 101. Similarly, in one example an inner diameter of the second elongated hollow member 114 is constant and is within about 30% of an inner diameter of the lumen 116 of the distal portion 118 of the tube 101. In one particular example, the inner diameter of the first elongated hollow member 108 is equal to or greater than the inner diameter of the lumen 110 of the proximal portion 112 of the tube 101, and the inner diameter of the second elongated hollow member 114 is equal to or greater than the inner diameter of the lumen 116 of the distal portion 118 of the tube 101. The arrangements described above may help to ensure that the device 100 does not significantly alter the flow rate of a fluid through the tube 101 when the device 100 is installed onto the tube 101. Further, these arrangements may help to ensure that the device 100 does not clog due to debris or blood clots when in use.

In one example, the first connector 104 and the second connector interact to provide a watertight seal between the first connector 104 and the second connector 106 when the first connector 104 is removably coupled to the second connector 106. In one particular example, the first connector 104 includes a first water tight component 124, and the second connector 106 includes a second water tight component 126 configured to interact with the first water tight component 124 when the first connector 104 is removably coupled to the second connector 106 to thereby provide the watertight seal between the first connector 104 and the second connector 106. In one example, the first water tight component 124 comprises an O-ring, and the second water tight component 126 comprises a cavity configured to receive the O-ring. In another example, the second water tight component 126 comprises an O-ring, and the first water tight component 124 comprises a cavity configured to receive the O-ring. Other water tight components including compression and surface contact coupling to maintain the flow through the elongated hollow members of both connectors are possible as well.

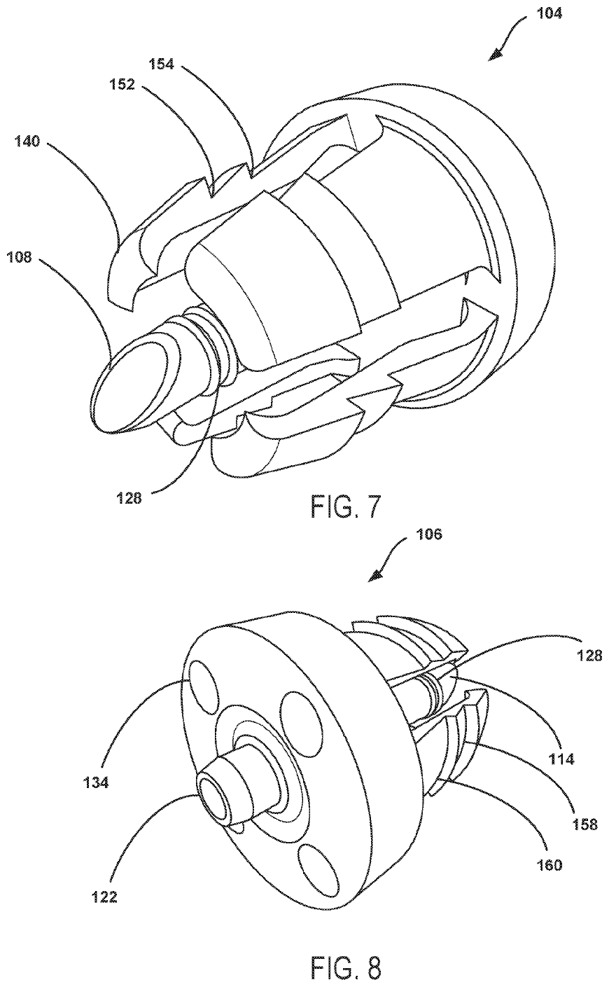

In one example, as shown in FIG. 7, the first elongated hollow member 108 includes one or more ridges 128 extending away from an exterior surface of the first elongated hollow member 108. As shown in FIG. 8, the second elongated hollow member 114 may similarly include one or more ridges 128 extending away from an exterior surface of the second elongated hollow member 114. The one or more ridges 128 provides increased friction within the lumen 110 of the proximal portion 112 of the tube 101 and the lumen 116 of the distal portion 118 of the tube 101. Further, the one or more ridges 128 may prevent leaking in between the first elongated hollow member 108 and the lumen 110 of the proximal portion 112 of the tube 101 and between the second elongated hollow member 114 and the lumen 116 of the distal portion 118 of the tube 101. Further, the one or more ridges 128 may provide a pinch-point for the clamping force to the proximal portion 112 of the tube 101 and a pinch-point for the clamping force to the distal portion 118 of the tube 101.

Importantly, the force required to separate the first elongated hollow member 108 from the lumen 110 of the proximal portion 112 of the tube 101 and the force required to separate the second elongated hollow member 114 from the lumen 116 of the distal portion 118 of the tube 101 must be greater than the force required to separate the first connector 104 from the second connector 106. In one example, an axial and bending force is needed to disconnect the second connector 106 from the first connector 104 about the central axis and center point between the two connectors. In such an example, the axial force required to disconnect the second connector 106 from the first connector 104 ranges from about 2N to 67N, and preferably from about 15N to about 20N. Further, in such an example the bending force needed to disconnect the second connector 106 from the first connector 104 ranges from about 0.2 Nm to about 4 Nm, and preferably from about 0.7 Nm to about 1 Nm.

As shown in FIGS. 7-8, in one example the first elongated hollow member 108 and the second elongated hollow member 114 each include an angled tip 130. The angled tip 130 allows for easy insertion into the lumen 110 of the proximal portion 112 of the tube 101 and the lumen 116 of the distal portion 118 of the tube 101 by providing a sharp point without reducing the diameter of the first elongated hollow member 108 and the second elongated hollow member 114 (similar to a syringe needle).

The first connector 104 may be removably coupled to the second connector 106 in a variety of ways. In one example, the first connector 104 is removably coupled to the second connector 106 via a magnetic connection. In such an example, as shown in FIG. 5, the first connector 104 includes one or more first magnets 132, and the second connector 106 includes one or more second magnets 134 configured to interact with the one or more first magnets 132 to thereby removably couple the first connector 104 to the second connector 106.

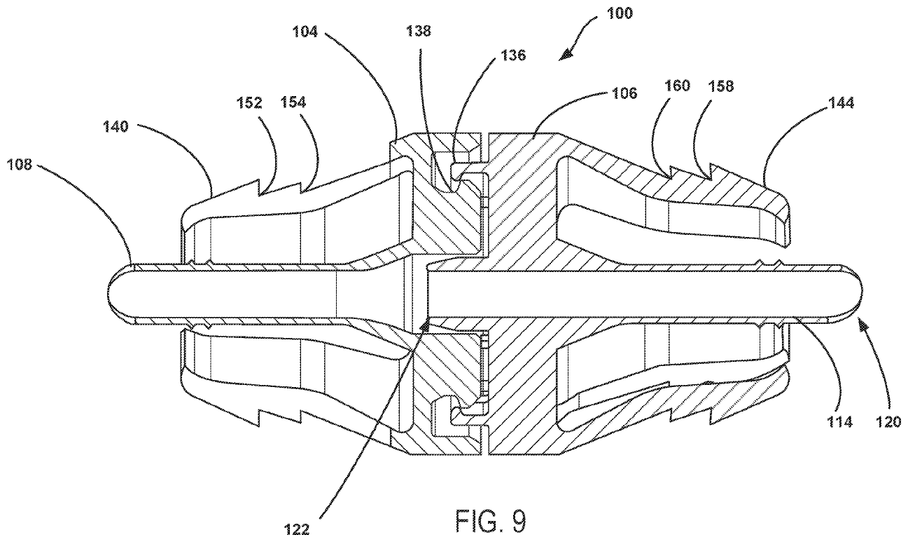

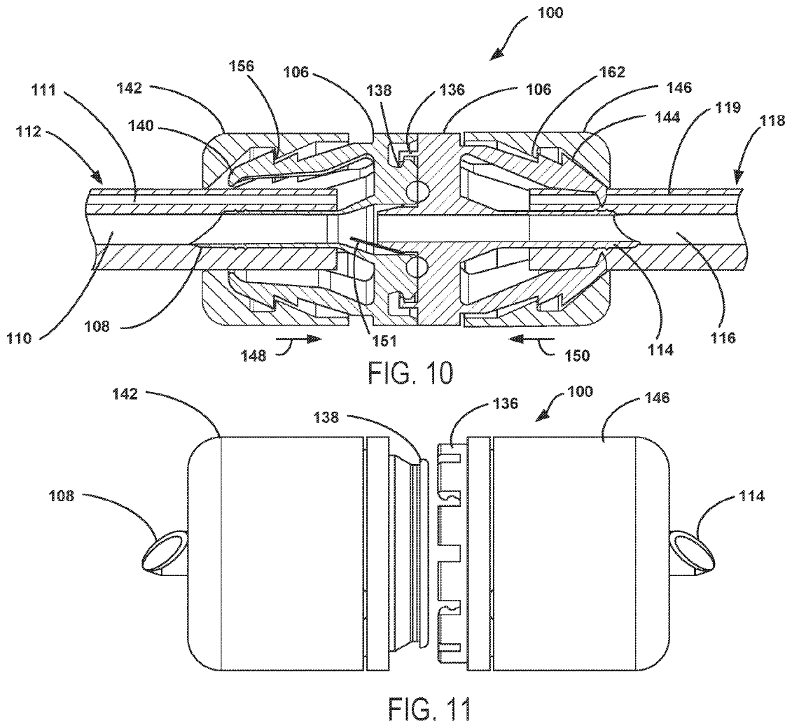

In another example, the first connector 104 is removably coupled to the second connector 106 via a mechanical connection. In one such example, as shown in FIGS. 9-10, the first connector 104 includes a plurality of arms 136, and the second connector 106 includes a coupling mechanism 138 with which the plurality of arms 136 are configured to interact to thereby removably couple the first connector 104 to the second connector 106. In another example, an inverse to the configuration described above in relation to FIGS. 9-10 is also possible. In particular, the second connector 106 may include the plurality of arms 136, and the first connector 104 includes the coupling mechanism 138 with which the plurality of arms 136 are configured to interact to thereby removably couple the first connector 104 to the second connector 106. In one particular example, the coupling mechanism 138 may comprise a ring with a lip over which the plurality of arms 136 are configured to be positioned, as shown in FIGS. 10-11. Other temporary mechanism couplings between the first connector 104 and the second connector 106 are possible as well.

In one example, as shown in FIGS. 7-11, the first connector 104 includes a first plurality of arms 140 positioned circumferentially around the first elongated hollow member 108, and a first cap 142 positioned over the first plurality of arms 140. In one example, the first cap 142 is configured to move axially along a longitudinal axis of the tube 101. As further shown in the embodiments of FIGS. 7-11, the second connector 106 a second plurality of arms 144 positioned circumferentially around the second elongated hollow member 114 and a second cap 146 positioned over the second plurality of arms 144. In one example, the second cap 146 is configured to move axially along the longitudinal axis of the tube 101. In use, the first plurality of arms 140 are configured to transition from a first position to a second position when the first cap 142 moves with respect to the first plurality of arms 140 (e.g., in a distal direction 148 as shown in FIG. 10 as a non-limiting example), and the first plurality of arms 140 are configured to provide the clamping force to the proximal portion 112 of the tube 101 when the first plurality of arms 140 are in the second position. As such, the first plurality of arms 140 are closer to the first elongated hollow member 108 in the second position than in the first position. Similarly, the second plurality of arms 144 are configured to transition from a first position to a second position when the second cap 146 moves with respect to the second plurality of arms 144 (e.g., in a proximal direction 150 as shown in FIG. 10 as a non-limiting example), and the second plurality of arms 144 are configured to provide the clamping force to the distal portion 118 of the tube 101 when the second plurality of arms 144 are in the second position. As such, the second plurality of arms 144 are closer to the second elongated hollow member 114 in the second position than in the first position. In one example, the first plurality of arms 140 and the second plurality of arms 144 each have a sharp tip such that all the clamping force is concentrated into a single radial section of the tube 101. This ensures the maximum pinch force is achieved and prevents the force from being distributed along the length of the tube 101.

In one example, each of the first plurality of arms 140 includes a first indent 152 and a second indent 154, and the first cap 142 includes a protrusion 156 configured to mate with the first indent 152 when the first plurality of arms 140 are in the first position and the protrusion 156 is configured to mate with the second indent 154 when the first plurality of arms 140 are in the second position. Similarly, in one example each of the second plurality of arms 144 includes a first indent 158 and a second indent 160, and the second cap 146 includes a protrusion 162 configured to mate with the first indent 158 when the second plurality of arms 144 are in the first position and the protrusion 162 is configured to mate with the second indent 160 when the second plurality of arms 144 are in the second position. FIG. 10 illustrates the first plurality of arms 140 in the first position with the protrusion 156 mating with the first indent 152 of the first plurality of arms 140. FIG. 10 further illustrates the second plurality of arms 144 in the second position with the protrusion 162 mating with the second indent 160 of the second plurality of arms 144 and providing the clamping force to the distal portion 118 of the tube 101.

In another example, the first cap 142 is threaded to provide a plurality of positions for the first plurality of arms 140 between the first position and the second position. Similarly, in one example the second cap 146 is threaded to provide a plurality of positions for the second plurality of arms 144 between the first position and the second position.

In one example, the first plurality of arms 140 are locked in the second position once the first plurality of arms 140 transition from the first position to the second position such that the first plurality of arms 140 cannot transition from the second position back to the first position. Similarly, in one example the second plurality of arms 144 are locked in the second position once the second plurality of arms 144 transition from the first position to the second position such that the second plurality of arms 144 cannot transition from the second position back to the first position.

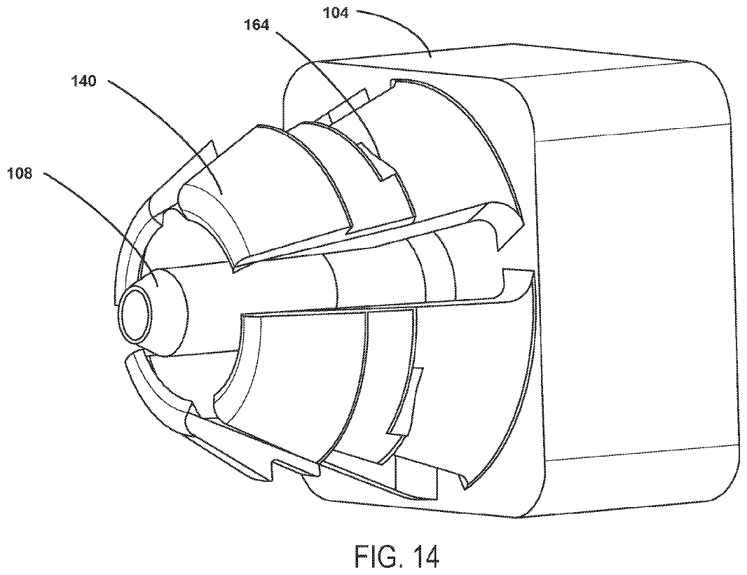

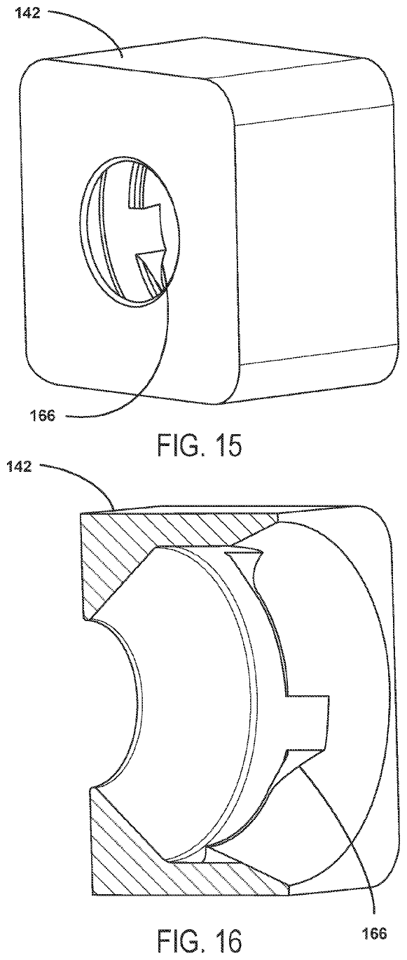

In another example, the first plurality of arms 140 can transition from the second position to the first position to thereby release the clamping force to the proximal portion 112 of the tube 101, and the second plurality of arms 144 can transition from the second position to the first position to thereby release the clamping force to the distal portion 118 of the tube 101. In one such example, as shown in FIGS. 14-16, each of the first plurality of arms 140 includes a first ramp 164, the first cap 142 includes a second ramp 166, and a rotation of the first cap 142 with respect to the first plurality of arms 140 causes the first ramp 164 to interact with the second ramp 166 to thereby transition the first plurality of arms 140 from the second position to the first position. Similarly, in one example, each of the second plurality of arms 144 includes a first ramp 164, the second cap includes a second ramp 166, and a rotation of the second cap 146 with respect to the second plurality of arms 144 causes the first ramp 164 to interact with the second ramp 166 to thereby transition the second plurality of arms 144 from the second position to the first position.

In one example, as shown in FIG. 10, the device further includes a flow prevention mechanism 151 positioned in the first connector 104. The flow prevention mechanism 151 is configured to prevent a fluid from exiting the first elongated hollow member 108 when the first connector 104 is disconnected from the second connector 106. In one particular example, as shown in FIG. 10, the flow prevention mechanism 151 comprises a lever that is configured to rotate into a first position when the second connector 106 is removably coupled to the first connector 104. As shown in FIG. 10, in the first position the flow prevention mechanism 151 does not occlude the first elongated hollow member 108 such that fluid can flow freely from the lumen 110 of the proximal portion 112 of the tube 101, through the first elongated hollow member 108, through the second elongated hollow member 114, and through the lumen 116 of the distal portion 118 of the tube 101. When the second connector 106 is disconnected from the first connector, the flow prevention mechanism 151 is configured to automatically rotate into a second position in which the flow prevention mechanism 151 occludes the first elongated hollow member 108 to thereby prevent fluid from exiting the first elongated hollow member 108.

In another example, the flow prevention mechanism 151 comprises a plurality of arms that are pushed apart into a first position when the second connector 106 is removably coupled to the first connector 104. In the first position the flow prevention mechanism 151 does not occlude the first elongated hollow member 108 such that fluid can flow freely from the lumen 110 of the proximal portion 112 of the tube 101, through the first elongated hollow member 108, through the second elongated hollow member 114, and through the lumen 116 of the distal portion 118 of the tube 101. When the second connector 106 is disconnected from the first connector, the plurality of arms of the flow prevention mechanism 151 are configured to automatically rotate and overlap with one another into a second position in which the flow prevention mechanism 151 occludes the first elongated hollow member 108 to thereby prevent fluid from exiting the first elongated hollow member 108. Other flow prevention mechanisms are possible as well.

In use, in addition to providing the clamping force around the tube 101, the first cap 142 and the second cap 146 provide a smooth round surface to prevent irritation to the skin of the patient and prevent bacterial buildup. When in their final position, the first cap 142 and the second cap 146 together conceal all internal components of the device 100. This ensures that the device 100 cannot be tampered with by the patient, and it also helps to prevent bacteria from reaching the inside of the tube 101. The first cap 142 and/or the second cap 146 may possess an additional O-ring or other sealing mechanism to further prevent bacteria or fluid from entering the inside of the tube 101.

As described above, the device 100 provides a clamping force to the proximal portion 112 of the tube 101 and a clamping force to the distal portion 118 of the tube 101. These clamping forces provides a strong grip between the first connector 104 and the proximal portion 112 of the tube 101 and between the second connector 106 and the distal portion 118 of the tube. Further, in some embodiments the clamping force to the proximal portion 112 of the tube 101 occludes a second lumen 111 of the proximal portion 112 of the tube 101. Such an arrangement ensures that the balloon 113 remains inflated even if the first connector 104 is separated from the second connector 106. In one example, the clamping force to the proximal portion 112 of the tube 101 is symmetrical with respect to a longitudinal axis of the tube 101. The symmetrical nature of such a clamping force helps to ensure that no matter where the second lumen 111 of the proximal portion 112 of the tube 101 is oriented with respect to the central axis of the tube 101, the symmetrically applied clamping force will occlude the second lumen 111. Further, the clamping force to the distal portion 118 of the tube 101 may occlude a second lumen 119 of the distal portion 118 of the tube 101. Such an arrangement may prevent bacteria and/or stale water from forming in the second lumen 119 of the distal portion 118 of the tube 101. In one example, the clamping force to the distal portion 118 of the tube 101 is symmetrical with respect to a longitudinal axis of the tube 101. The symmetrical nature of such a clamping force helps to ensure that no matter where the second lumen 119 of the distal portion 118 of the tube 101 is oriented with respect to the central axis of the tube 101, the symmetrically applied clamping force will occlude the second lumen 119.

Various clamping mechanisms for providing the clamping force to the tube 101 are possible. As described above, in one example the first connector 104 includes a first plurality of arms 140 that provide the clamping force, and the second connector 106 includes a second plurality of arms 144 that provide the clamping force. In another example, a ratcheting mechanism may be used to provide the clamping force. In another example, a metal cord or drawstring may be used to provide the clamping force. In another example, a zip tie may be used to provide the clamping force. In another example, a hose clamp may be used to provide the clamping force. Other examples are possible as well.

As described above, the tube 101 that is used with the device 100 may comprise one of a urinary catheter, an intravenous (IV) line, a spinal tap tubing, a nasogastric (NG) tube, a suprapubic catheter, or a gastrostomy tube, as non-limiting examples. In some examples, the tube 101 may include a single lumen. In another example, the tube 101 may include two lumens (e.g., the lumen 110 and the second lumen 111). In another example, the tube 101 may include three lumens including a drainage lumen, an inflation lumen, and a lumen for irrigating the bladder. Other numbers of lumens for the tube 101 are possible as well, and the above examples are not meant to be limiting.

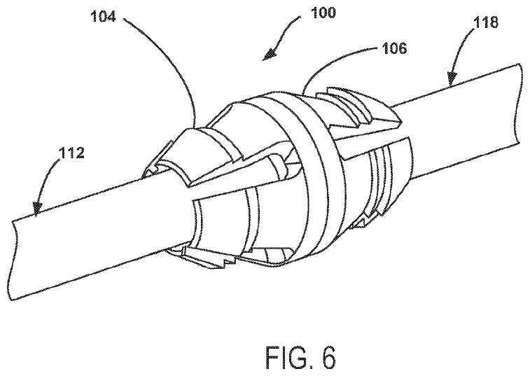

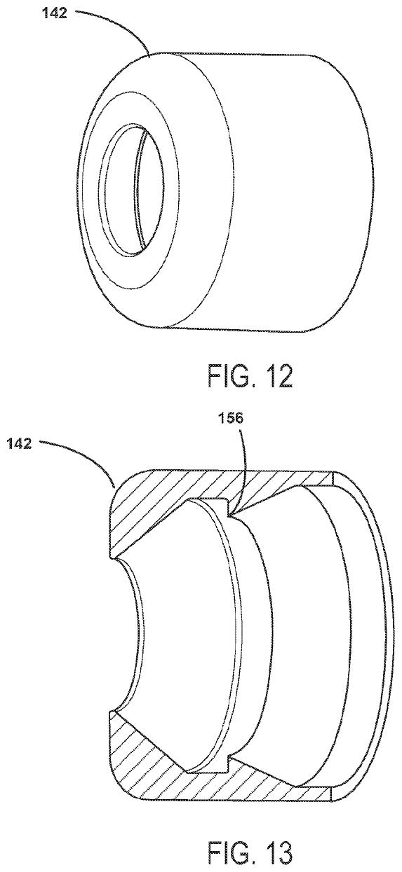

In one example, the present disclosure provides a kit comprising the device of any one of the embodiments described above with relation to FIGS. 1-16, and a urinary catheter 103. As shown in FIGS. 1-4, the urinary catheter 103 includes a tube 101 having a distal end 105 and a proximal end 107 opposite the distal end 105. As shown in FIG. 5, the tube 101 includes a lumen 110 (which acts as a drainage lumen) and a second lumen 111 (which acts as an inflation lumen). The urinary catheter 103 further includes a balloon 113 positioned near the proximal end 107 of the tube 101. The second lumen 111 (inflation lumen) is in communication with an interior of the balloon 113. The urinary catheter 103 further includes a drainage port 109 in fluid communication with the lumen 110 (drainage lumen). The drainage port 109 is configured to be connected to a bag or other receptacle to collect urine from the patient. The urinary catheter 103 further includes an inflation port 115 in fluid communication with the second lumen (inflation lumen). The inflation port 115 is configured to be coupled to a syringe or other device to provide saline, water, or other material through the second lumen to thereby inflate the balloon 113 once the balloon is positioned in the bladder of the patient.

In one example, the present disclosure provides a kit comprising the device of any one of the embodiments described above with relation to FIGS. 1-16, and a drainage cap 168 configured to be removably coupled to the first connector 104. The drainage cap 168 prevents fluid from exiting the lumen 110 of the proximal portion 112 of the tube 101 through the first elongated hollow member 108.

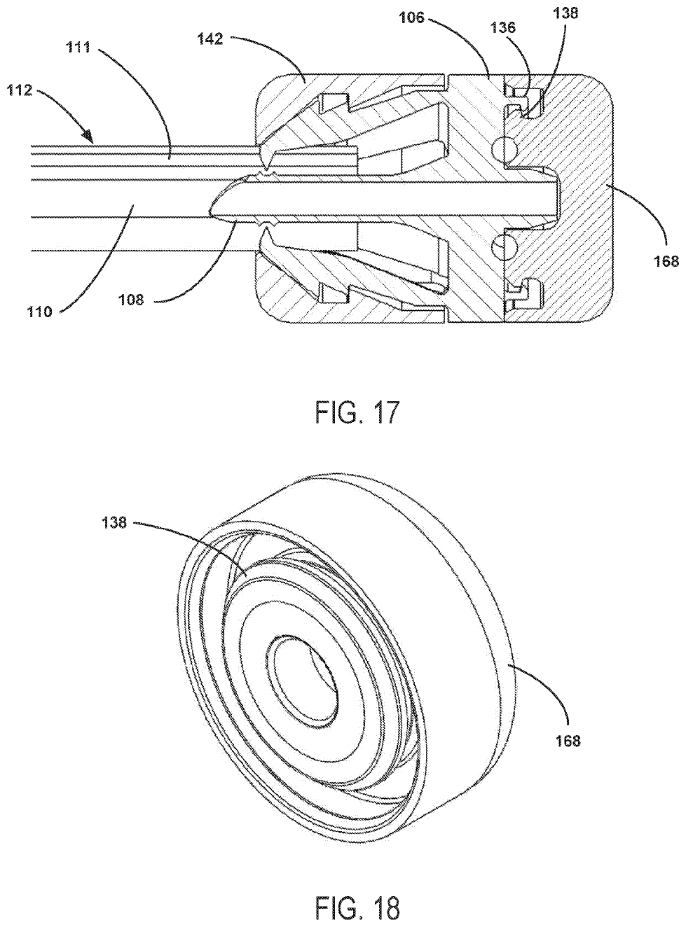

FIG. 17 illustrates a side cross-sectional view of the first connector 104 removably coupled to the drainage cap 168. FIG. 18 illustrates a perspective view of the drainage cap. Similar to the removable coupling between the first connector 104 and the second connector 106 discussed above, the first connector 104 may be removably coupled to the drainage cap 168 in a variety of ways. In one example, the first connector 104 is removably coupled to the drainage cap 168 via a magnetic connection. In such an example, the first connector 104 includes one or more first magnets 132, and the drainage cap 168 includes one or more second magnets configured to interact with the one or more first magnets 132 to thereby removably couple the first connector 104 to the drainage cap 168.

In another example, the first connector 104 is removably coupled to the drainage cap 168 via a mechanical connection. In one such example, as shown in FIGS. 17-18, the first connector 104 includes a plurality of arms 136, and the drainage cap 168 includes a coupling mechanism 138 with which the plurality of arms 136 are configured to interact to thereby removably couple the first connector 104 to the drainage cap 168. In another example, an inverse to the configuration described above in relation to FIGS. 17-18 is also possible. In particular, the drainage cap 168 may include the plurality of arms 136, and the first connector 104 includes the coupling mechanism 138 with which the plurality of arms 136 are configured to interact to thereby removably couple the first connector 104 to the drainage cap 168. In one particular example, the coupling mechanism 138 may comprise a ring with a lip over which the plurality of arms 136 are configured to be positioned, as shown in FIGS. 17-18. Other temporary mechanism couplings between the first connector 104 and the drainage cap 168 are possible as well.

In use, the drainage cap 168 is removably coupled to the first connector 104 (in essentially the same way as the second connector 106) and creates a sealed cavity in which fluid cannot pass. The drainage cap 168 may be used for patients that may want to stay catheterized but do not want their urine to drain into a bag. For example, an active patient who wants to go on a run may disconnect the second connector 106 of the device 100 and connect the drainage cap 168. When ready, the patient can disconnect the drainage cap 168 and reconnect the second connector 106, resuming urine drainage.

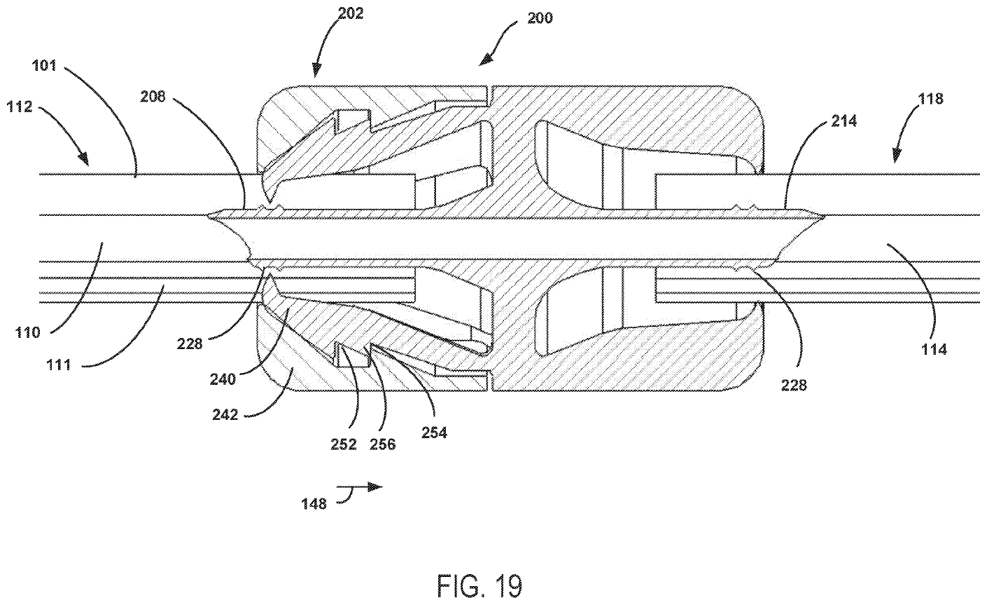

FIG. 19 illustrates another example device 200 according to an example embodiment. As shown in FIG. 19, the device 200 includes a first elongated hollow member 208 configured to be positioned at least partially within a lumen 110 of a proximal portion 112 of a tube 101. The device 200 further includes a clamping mechanism 202 configured to provide a clamping force to the proximal portion 112 of the tube 101. The device 200 also includes a second elongated hollow member 214 configured to be positioned at least partially within a lumen 116 of a distal portion 118 of the tube 101. The second elongated hollow member 214 and the first elongated hollow member 208 are fluidically connected such that the lumen 110 of the proximal portion 112 of the tube 101 is fluidically connected with the lumen 116 of the distal portion 118 of the tube 101. Due to the clamping force applied to the proximal portion 112 of the tube 101 by the clamping mechanism 202, a force required to remove the first elongated hollow member 208 from the lumen 110 of the proximal portion 112 of the tube 101 is greater than a force required to remove the second elongated hollow member 214 from the lumen 116 of the distal portion 118 of the tube 101.

In one example, the first elongated hollow member 208 includes one or more ridges 228 extending away from an exterior surface of the first elongated hollow member 208. As shown in FIG. 19, the second elongated hollow member 114 may similarly include one or more ridges 228 extending away from an exterior surface of the second elongated hollow member 214. The one or more ridges 228 provides increased friction within the lumen 110 of the proximal portion 112 of the tube 101 and the lumen 116 of the distal portion 118 of the tube 101. Further, the one or more ridges 228 may prevent leaking in between the first elongated hollow member 108 and the lumen 110 of the proximal portion 112 of the tube 101 and between the second elongated hollow member 214 and the lumen 116 of the distal portion 118 of the tube 101. Further, the one or more ridges 228 may provide a pinch-point for the clamping force to the proximal portion 112 of the tube 101.

In one example, the clamping force applied by the clamping mechanism 202 to the proximal portion 112 of the tube 101 occludes a second lumen 111 of the proximal portion 112 of the tube 101. Such an arrangement ensures that the balloon 113 remains inflated even if the first connector 104 is separated from the second connector 106. In one example, the clamping force to the proximal portion 112 of the tube 101 is symmetrical with respect to a longitudinal axis of the tube 101. The symmetrical nature of such a clamping force helps to ensure that no matter where the second lumen 111 of the proximal portion 112 of the tube 101 is oriented with respect to the central axis of the tube 101, the symmetrically applied clamping force will occlude the second lumen 111.

In one example, an inner diameter of the first elongated hollow member 208 is constant and is within about 30% of an inner diameter of the lumen 110 of the proximal portion 112 of the tube 101. Similarly, in one example an inner diameter of the second elongated hollow member 214 is constant and is within about 30% of an inner diameter of the lumen 116 of the distal portion 118 of the tube 101. In one particular example, the inner diameter of the first elongated hollow member 208 is equal to or greater than the inner diameter of the lumen 110 of the proximal portion 112 of the tube 101, and the inner diameter of the second elongated hollow member 214 is equal to or greater than the inner diameter of the lumen 116 of the distal portion 118 of the tube 101. The arrangements described above may help to ensure that the device 200 does not significantly alter the flow rate of a fluid through the tube 101 when the device 200 is installed onto the tube 101. Further, these arrangements may help to ensure that the device 200 does not clog due to debris or blood clots when in use.

In an example, as shown in FIG. 19, the clamping mechanism 202 comprises a plurality of arms 240 positioned circumferentially around the first elongated hollow member 208, and a cap 242 positioned over the plurality of arms 240. The plurality of arms 240 and the cap 242 may be similarly configured to the embodiments described in relation to FIGS. 7-11 above.

In use, the plurality of arms 240 are configured to transition from a first position to a second position when the cap 242 moves with respect to the first plurality of arms 140 (e.g., in a distal direction 148 as shown in FIG. 19 as a non-limiting example), and plurality of arms 240 are configured to provide the clamping force to the proximal portion 112 of the tube 101 when the plurality of arms 240 are in the second position. As such, the plurality of arms 240 are closer to the first elongated hollow member 208 in the second position than in the first position. In one example, the plurality of arms 240 each have a sharp tip such that all the clamping force is concentrated into a single radial section of the tube 101. This ensures the maximum pinch force is achieved and prevents the force from being distributed along the length of the tube 101.

In one example, each of the plurality of arms 240 includes a first indent 252 and a second indent 254, and the cap 242 includes a protrusion 256 configured to mate with the first indent 252 when the plurality of arms 240 are in the first position and the protrusion 256 is configured to mate with the second indent 254 when the plurality of arms 240 are in the second position. FIG. 19 illustrates the plurality of arms 240 in the second position with the protrusion 156 mating with the second indent 254 of the plurality of arms 240 and providing the clamping force to the proximal portion 112 of the tube 101.

In another example, the cap 242 is threaded to provide a plurality of positions for the plurality of arms 240 between the first position and the second position.

In one example, the plurality of arms 240 are locked in the second position once the plurality of arms 240 transition from the first position to the second position. In another example, the plurality of arms 240 can transition from the second position to the first position to thereby release the clamping force to the proximal portion 112 of the tube 101. In one such example, the cap 242 may be configured similar to the cap as shown and described above in relation to FIGS. 14-16.

In one example, the present disclosure provides a kit comprising the device of any one of the embodiments described above with relation to FIG. 19, and a urinary catheter 103. As shown in FIGS. 1-4, the urinary catheter 103 includes a tube 101 having a distal end 105 and a proximal end 107 opposite the distal end 105. As shown in FIG. 5, the tube 101 includes a lumen 110 (which acts as a drainage lumen) and a second lumen 111 (which acts as an inflation lumen). The urinary catheter 103 further includes a balloon 113 positioned near the proximal end 107 of the tube 101. The second lumen 111 (inflation lumen) is in communication with an interior of the balloon 113. The urinary catheter 103 further includes a drainage port 109 in fluid communication with the lumen 110 (drainage lumen). The drainage port 109 is configured to be connected to a bag or other receptacle to collect urine from the patient. The urinary catheter 103 further includes an inflation port 115 in fluid communication with the second lumen (inflation lumen). The inflation port 115 is configured to be coupled to a syringe or other device to provide saline, water, or other material through the second lumen to thereby inflate the balloon 113 once the balloon is positioned in the bladder of the patient.

FIG. 20 is a block diagram of an example method for installing a urinary catheter breakaway device. Method 300 shown in FIG. 20 presents an embodiment of a method that could be used by the device 100 as described above in relation to FIGS. 1-18, as examples. Method 300 may include one or more operations, functions, or actions as illustrated by one or more of blocks 302-312. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 302, the method 300 includes cutting a urinary catheter 103 between a proximal end 107 and a distal end 105 of the urinary catheter 103 to thereby define a proximal portion 112 and a distal portion 118 of the urinary catheter 103. At block 304, the method 300 includes positioning a first elongated hollow member 108 of a first connector 104 at least partially within a lumen 110 of the proximal portion 112 of the urinary catheter 103. At block 306, the method 300 includes positioning a second elongated hollow member 114 of a second connector 106 at least partially within a lumen 116 of the distal portion 118 of the urinary catheter 103. At block 308, the method 300 includes providing a clamping force to the proximal portion 112 of the urinary catheter 103. At block 310, the method 300 includes providing a clamping force to the distal portion 118 of the urinary catheter 103. At block 312, the method 300 includes removably coupling the first connector 104 to the second connector 106, where the lumen 110 of the proximal portion 112 of the urinary catheter 103 is fluidically connected with the lumen 116 of the distal portion 118 of the urinary catheter 103 when the first connector 104 is removably coupled to the second connector 106.

In one example, the method 300 further includes (a) applying a pinching force to the proximal portion 112 of the urinary catheter 103 and the distal portion 118 of the urinary catheter 103 prior to cutting the urinary catheter 103, and (b) removing the pinching force after removably coupling the first connector 104 to the second connector 106. Such an arrangement may help prevent the fluid in the balloon 113 from draining before the device 100 is installed. In one example, such a pinching force may be applied via a forceps prior to cutting the urinary catheter 103.

In one example, the clamping force to the proximal portion 112 of the urinary catheter 103 occludes a second lumen 111 of the proximal portion 112 of the urinary catheter 103, and the clamping force to the distal portion 118 of the urinary catheter 103 occludes a second lumen 117 of the distal portion 118 of the urinary catheter 103.

FIG. 21 is a block diagram of another example method for installing a urinary catheter breakaway device. Method 400 shown in FIG. 21 presents an embodiment of a method that could be used by the device 200 as described above in relation to FIG. 19, as examples. Method 400 may include one or more operations, functions, or actions as illustrated by one or more of blocks 402-408. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 402, the method 400 includes cutting a urinary catheter 103 between a proximal end 107 and a distal end 105 of the urinary catheter 103 to thereby define a proximal portion 112 and a distal portion 118 of the urinary catheter 103. At block 304, the method 300 includes positioning a first elongated hollow member 208 at least partially within a lumen 110 of the proximal portion 112 of the urinary catheter 103. At block 406, the method 300 includes positioning a second elongated hollow member 214 at least partially within a lumen 116 of the distal portion 118 of the urinary catheter 103. At block 408, the method 400 includes providing a clamping force to the proximal portion 112 of the urinary catheter 103 such that a force required to remove the first elongated hollow member 208 from the lumen 110 of the proximal portion 112 of the urinary catheter 103 is greater than a force required to remove the second elongated hollow member 214 from the lumen 116 of the distal portion 118 of the urinary catheter 103.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A device comprising:
a first connector including a first elongated hollow member configured to be positioned at least partially within a lumen of a proximal portion of a tube, wherein the first connector provides a clamping force to the proximal portion of the tube; and
a second connector configured to be removably coupled to the first connector, the second connector including a second elongated hollow member configured to be positioned at least partially within a lumen of a distal portion of the tube, wherein the second connector provides a clamping force to the distal portion of the tube, wherein the proximal portion of the tube is separated from the distal portion of the tube when the first connector is disconnected from the second connector, and wherein the lumen of the proximal portion of the tube is fluidically connected with the lumen of the distal portion of the tube when the first connector is removably coupled to the second connector.

2. The device of claim 1, wherein the second elongated hollow member comprises a first portion configured to be positioned at least partially within the lumen of the distal portion of the tube, and wherein the second elongated hollow member comprises a second portion configured to be positioned at least partially within the first elongated hollow member when the first connector is removably coupled to the second connector.

3. The device of claim 1, wherein the first elongated hollow member comprises a first portion configured to be positioned at least partially within the lumen of the proximal portion of the tube, and wherein the first elongated hollow member comprises a second portion configured to be positioned at least partially within the second elongated hollow member when the first connector is removably coupled to the second connector.

4. The device of claim 1, wherein the first connector and the second connector interact to provide a watertight seal between the first connector and the second connector when the first connector is removably coupled to the second connector.

5. The device of claim 1, wherein the first elongated hollow member includes one or more ridges extending away from an exterior surface of the first elongated hollow member, and wherein the second elongated hollow member includes one or more ridges extending away from an exterior surface of the second elongated hollow member.

6. The device of claim 1, wherein the first connector includes one or more first magnets, and wherein the second connector includes one or more second magnets configured to interact with the one or more first magnets to thereby removably couple the first connector to the second connector.

7. The device of claim 1, wherein the first connector includes a plurality of arms, and wherein the second connector includes a coupling mechanism with which the plurality of arms are configured to interact to thereby removably couple the first connector to the second connector.

8. The device of claim 1, wherein the clamping force to the proximal portion of the tube occludes a second lumen of the proximal portion of the tube.

9. The device of claim 1, wherein the clamping force to the distal portion of the tube occludes a second lumen of the distal portion of the tube.

10. The device of claim 1,
wherein the first connector includes:
a first plurality of arms positioned circumferentially around the first elongated hollow member; and
a first cap positioned over the first plurality of arms, and
wherein the second connector includes:
a second plurality of arms positioned circumferentially around the second elongated hollow member; and
a second cap positioned over the second plurality of arms.

11. The device of claim 10, wherein the first plurality of arms are configured to transition from a first position to a second position when the first cap moves with respect to the first plurality of arms, wherein the first plurality of arms are configured to provide the clamping force to the proximal portion of the tube when the first plurality of arms are in the second position, wherein the second plurality of arms are configured to transition from a first position to a second position when the second cap moves with respect to the second plurality of arms, and wherein the second plurality of arms are configured to provide the clamping force to the distal portion of the tube when the second plurality of arms are in the second position.

12. The device of claim 11, wherein each of the first plurality of arms includes a first indent and a second indent, wherein the first cap includes a protrusion configured to mate with the first indent when the first plurality of arms are in the first position, wherein the protrusion is configured to mate with the second indent when the first plurality of arms are in the second position, wherein each of the second plurality of arms includes a first indent and a second indent, wherein the second cap includes a protrusion configured to mate with the first indent when the second plurality of arms are in the first position, and wherein the protrusion is configured to mate with the second indent when the second plurality of arms are in the second position.

13. The device of claim 11, wherein the first plurality of arms are locked in the second position once the first plurality of arms transition from the first position to the second position, and wherein the second plurality of arms are locked in the second position once the second plurality of arms transition from the first position to the second position.

14. The device of claim 13, wherein the first plurality of arms can transition from the second position to the first position to thereby release the clamping force to the proximal portion of the tube, and wherein the second plurality of arms can transition from the second position to the first position to thereby release the clamping force to the distal portion of the tube.

15. The device of claim 14, wherein each of the first plurality of arms includes a first ramp, wherein the first cap includes a second ramp, wherein a rotation of the first cap with respect to the first plurality of arms causes the first ramp to interact with the second ramp to thereby transition the first plurality of arms from the second position to the first position, wherein each of the second plurality of arms includes a first ramp, wherein the second cap includes a second ramp, and wherein a rotation of the second cap with respect to the second plurality of arms causes the first ramp to interact with the second ramp to thereby transition the second plurality of arms from the second position to the first position.

16. The device of claim 1, wherein a diameter of the first elongated hollow member is constant and is within about 30% of a diameter of the lumen of the proximal portion of the tube, and wherein a diameter of the second elongated hollow member is constant and is within about 30% of a diameter of the lumen of the distal portion of the tube.

17. The device of claim 1, wherein the clamping force to the proximal portion of the tube is symmetrical with respect to a longitudinal axis of the tube.

18. The device of claim 1, wherein the clamping force to the distal portion of the tube is symmetrical with respect to a longitudinal axis of the tube.

19. The device of claim 1, further comprising a flow prevention mechanism positioned in the first connector, wherein the flow prevention mechanism is configured to prevent a fluid from exiting the first hollow elongated member when the first connector is disconnected from the second connector.

20.-57. (canceled)

58. A method for installing the device of claim 1, the method comprising:
cutting a urinary catheter between a proximal end and a distal end of the urinary catheter to thereby define a proximal portion and a distal portion of the urinary catheter;
positioning the first elongated hollow member of the first connector at least partially within a lumen of the proximal portion of the urinary catheter;
positioning the second elongated hollow member of the second connector at least partially within a lumen of the distal portion of the urinary catheter;
providing a clamping force to the proximal portion of the urinary catheter;
providing a clamping force to the distal portion of the urinary catheter; and
removably coupling the first connector to the second connector, wherein the lumen of the proximal portion of the urinary catheter is fluidically connected with the lumen of the distal portion of the urinary catheter when the first connector is removably coupled to the second connector.

59.-61. (canceled)
